(19) United States
(12) Patent Application Publication
(10) Pub. No.: US 2011/0028456 A1
(43) Pub. Date: Feb. 3, 2011

(54) Solid Pharmaceutical Dosage Form

(75) Inventors: Amar Lulla, Mumbai (IN); Geena Malhotra, Mumbai (IN)

Correspondence Address: CONLEY ROSE, P.C., 5601 GRANITE PARKWAY, SUITE 750, PLANO, TX, 75024, US

(73) Assignee: CIPLA LIMITED, Mumbai (IN)

(21) Appl. No.: 12/812,432

(22) PCT Filed: Jan. 12, 2009
(86) PCT No.: PCT/GB09/00083

§ 371 Date: Sep. 23, 2010

(30) Foreign Application Priority Data
Jan. 11, 2008 (IN) 89/MUM/2008
Mar. 10, 2008 (IN) 489/MUM/2008
Mar. 24, 2008 (IN) 619/MUM/2008

(51) Int. Cl. A61K 31/551; A61K 31/522; A61K 31/536; A61K 31/4365; A61K 31/506; A61K 31/277; A61K 31/155; A61K 31/137; A61K 31/44; A61K 31/167

(52) U.S. Cl. 514/220; 514/263.38; 514/230.5; 514/301; 514/256; 514/523; 514/635; 514/653; 514/356; 514/630

(57) ABSTRACT

A pharmaceutical composition comprising a solid unit dosage form comprising: one or more of pharmaceutically active ingredients selected from valacyclovir, olanzapine, voriconazole, topotecan, artesunate, amodiaquine, guggulosterone, ramipril, telmisartan, tibolone, atorvastatin, simvastatin, amlodipine, ezetimibe, fenofibrate, tacrolimus, valgancyclovir, valsartan, clopidrogel, estradiol, trenbolone, efavirenz, metformin, pseudoephedrine, verapamil, felodipine, valproic acid/sodium valproate, mesalamine, hydrochlorothiazide, levosulpiride, nelfinavir, cefixime and cefpodoxime proxetil in combination with a water insoluble polymer and/or a water soluble polymer. Methods for making the pharmaceutical composition are also disclosed.

### CROSS-REFERENCE TO RELATED APPLICATIONS

This application is a filing under 35 U.S.C. 371 of International Application No. PCT/GB2009/000083 filed Jan. 12, 2009, entitled “Solid Pharmaceutical Dosage Form,” claiming priority of Indian Patent Application Nos. 89/MUM/2008 filed Jan. 11, 2008, 489/MUM/2008 filed Mar. 10, 2008, and 619/MUM/2008 filed Mar. 24, 2008, which applications are incorporated by reference herein in their entirety.

### FIELD OF INVENTION

The present invention relates to a hot-melt extruded pharmaceutical composition comprising a pharmaceutical active ingredient dispersed as fine particles in a water soluble or insoluble polymer or a combination of both the polymers and a method of preparation thereof.

### BACKGROUND OF INVENTION

Pharmaceutical formulations comprised of active compounds finely and homogenously dispersed in one or more polymeric carriers have been described as solid dispersions, glass solutions, molecular dispersions, and solid solutions. The term solid dispersion has been used as a general term to describe pharmaceutical preparations in which the active compound is dispersed in an inert excipient carrier in a size range from coarse to fine. Glass solution, molecular dispersion, and solid solution refer specifically to preparations in which amorphous forms of a crystalline active compound are formed in-situ and dispersed within the polymer matrix during the hot-melt extrusion process.

Many researchers have produced such preparations with various active compounds and polymeric carriers using hot-melt extrusion techniques. Rosenberg and Breitenbach have produced solid solutions by melt extruding the active substance in a nonionic form together with a salt and a polymer, such as polyvinylpyrrolidone (PVP), vinylpyrrolidinone/vinylacetate (PVPVA) copolymer, or a hydroxyalkylcellulose. Six, et al, Brewster, et al, Baert, et al, and Verreck, et al have produced solid dispersions of itraconazole with improved dissolution rates by hot-melt extrusion with various polymeric carriers including hydroxypropylmethylcellulose, Eudragit E100, PVPVA, and a combination of Eudragit E100 and PVPVA. Forster, et al. produced amorphous glass solutions with the poorly water soluble drugs indomethacin, nifedipine, and tolbutamide in PVP and PVPVA demonstrating improved dissolution compared with the crystalline forms. In this article, it is also seen that after storage of the extrudates at 25° C. and 75% relative humidity, only compositions containing indomethacin and polymer in a one to one ratio remained completely amorphous. Formulations of the remaining drugs and formulations with increased indomethacin concentration showed re-crystallization on storage. This re-crystallization was shown to significantly decrease the dissolution rate of the active. It should also be noted that stability studies were not performed at elevated temperatures in this study. It would be expected that elevated temperatures would increase the occurrence and extent of recrystallization. The previous reference reveals the inherent instability of amorphous dispersions produced by hot-melt extrusion techniques. Although many articles demonstrate the production of amorphous solid dispersions and the resulting improvement of drug dissolution rate, very few discuss the stability of such preparations on storage. From the work of Foster, et al. and an understanding of the thermodynamics of amorphous systems, it can be concluded that recrystallization of amorphous solid dispersion formulations on storage is a common problem. The amorphous state is thermodynamically metastable, and therefore it is expected that amorphous compounds will assume a stable crystalline conformation with time, as well as in response to perturbations such as elevations in temperature and exposure to moisture. In an extruded formulation, amorphous drug particles will agglomerate and crystallize with increasing storage time, elevated temperature, or exposure to moisture, essentially precipitating out of the carrier. This progression towards phase separation during storage results in a time dependant dissolution profile. A change in dissolution rate with time precludes the successful commercialization of a pharmaceutical product.

The difficulty of producing stable single phase amorphous dispersions of high drug loading can be seen from references such as those given above. The appearance of a second phase of the active compound on processing or on storage would result in a time dependent biphasic dissolution profile, and would therefore not be considered an acceptable pharmaceutical preparation.

Although there have been many reports of successful production of solid dispersions by hot-melt extrusion that show improved dissolution rates of poorly water soluble drugs, the absence of numerous marketed products based on this technology is evidence that stability problems remain a major obstacle for successful commercialization of such a pharmaceutical preparation.

There are several methods well known in the pharmaceutical literature for producing fine drug particles in the micro or nanometer size range. These methods can be divided into three primary categories: (1) mechanical micronization, (2) solution based phase separation, and (3) rapid freezing techniques.

There are many solution based phase separation processes documented in the pharmaceutical literature for producing micro and nano-sized drug particles. Some of the more commonly known processes are spray drying, emulsification/evaporation, solvent extraction, and complex coacervation. Some of the lesser-known processes are, for the sake of brevity, listed below along with their respective illustrating references: a) gas antisolvent precipitation (GAS)—(27) and WO 90/03782, EP 0437451; b) precipitation with a compressed antisolvent (PCA)—(28) and U.S. Pat. No. 5,874,029; c) aerosol solvent extraction system (ASES)—(29); d) evaporative precipitation into aqueous solution (EPAS)—(30) U.S. Patent Application 2004/0067251; e) supercritical antisolvent (SAS)—(31); f) solution-enhanced dispersion by supercritical fluids (SEDS)—(32); g) rapid expansion from supercritical to aqueous solutions (RESAS)—(33); and h) anti-solvent precipitation. Freezing techniques for producing micro or nano-sized drug particles are listed below along with their respective illustrating references: a) spray freezing into liquid (SFL)—(34) WO 02/060411, U.S. Patent Application 2003/054042; and b) ultra rapid freezing (URF)—(35). It should be noted that fine drug particles produced by solution-based phase separation or rapid freezing techniques are often amorphous in nature. These amorphous particles can be stabilized by complexation or coating during the production process with one or more excipient carriers having high melting points or glass transition temperatures. Stabilized amorphous fine drug particles can be formulated into the present preparation in the same manner as crystalline fine drug particles. The high shear of the hot-melt extrusion process will effectively deaggregate and disperse the amorphous drug particles (likely to be aggregated before extrusion due to high surface energy) into the stabilizing and non-solubilizing carrier thereby separating the aggregated particles into primary particles that are stabilized against aggregation and agglomeration on processing and storage by the carrier system. The excipient system with which the amorphous drug particles are complexed or coated will prevent recrystallization during hot-melt extrusion and storage of the amorphous drug-containing particle domains that are dispersed in the stabilizing and non-solubilizing carrier matrix. The benefit of this form of an amorphous dispersion compared to a traditional amorphous dispersion is that the formation of fine amorphous drug particles is not dependent on the solubility of the drug in the carrier system, since the amorphous drug particles are not formed in-situ by the solubilization of the crystalline drug particles by the carrier system.

It has been reported that fine drug particles produced by processes such as those listed above exhibit high surface energy resulting in strong cohesive forces between particles. It is known that powders of fine particles are likely to aggregate because the force of detachment is dependent on particle mass which is small in the case of fine particles. The forces of cohesion between individual fine particles are therefore greater than the forces of detachment, and thus particle aggregates form, hence the extent of aggregation is increased as particle size is reduced.

Aggregation of fine particles results in an increase in the apparent particle size, consequently, particle size reduction is somewhat negated. In order to achieve the full benefit of particle size reduction, i.e., accelerated dissolution rate, aggregates must be reduced to individual particles when dosed.

Particle agglomeration with storage also causes an increase in apparent particle size, and a corresponding decrease in dissolution rate. In the production of an ideal solid dosage form containing fine drug particles, aggregates would be separated and stabilized as individual particles by a carrier system during processing. The carrier system would also function to impede particle aggregation and agglomeration on storage at ambient and accelerated temperature and humidity conditions.

Prior art examples such as those mentioned above demonstrate the ongoing need for the advantageous properties of the present invention for the delivery of drug from a hot-melt extruded composition comprising fine drug particles.

WO 02/35991 discloses a process for producing spheronized pellets by hot-melt extrusion and spheronization.

WO 97/49384 discloses pharmaceutical formulations comprising a hot-melt extrudable mixture of a therapeutic compound and a high molecular weight polyethylene oxide (PEO) optionally containing polyethylene glycol as a plasticizer.

U.S. Patent Application 2004/0253314 discloses melt extrusion formulations comprising an active pharmaceutical ingredient and a methacrylate copolymer comprised of 40 to 75 weight % of radically copolymerized C₁₋₄ alkyl esters of acrylic acid or of methacrylic acid.

EP 1663183 discloses solid pharmaceutical dosage forms comprising a solid dispersion of at least one HIV protease inhibitor, at least one pharmaceutically acceptable water soluble polymer and at least one pharmaceutically acceptable surfactant wherein the water soluble polymer has a T_{g} (glass transition temperature) of at least about 50° C.

WO 2007/068615 discloses a pharmaceutical composition containing a solid suspension prepared by hot-melt extrusion isobutyric acid (2R,3S,4R,5R)-5-(4-amino-2-oxo-2H-pyrimidin-1-yl)-2-azido-3,4-bis-iso-butyryloxy-tetrahydro-furan-2-ylmethyl ester; hydrochloride salt (I) and a polyethylene glycol (PEG)/polypropylene glycol (PPG) block copolymer for the therapy of hepatitis C virus (HCV).

U.S. Patent Application 2007/0071813 discloses a process for preparing a pharmaceutical tablet composition wherein an active pharmaceutical ingredient and a water soluble poloxamer are processed by hot-melt extrusion before mixing with other ingredients.

### OBJECT OF THE INVENTION

It is an object of the present invention to provide high drug loading of fine drug particles, preferably in an oral composition comprising one or more active pharmaceutical ingredients.

Another object of the present invention is to provide pharmaceutical formulations comprised of active compounds finely and homogenously dispersed in one or more polymeric carriers that are produced by hot-melt extrusion techniques.

Another object of the present invention is to provide a pharmaceutical composition with ease of manufacture.

The present invention addresses the problem of physical instability of traditional solid dispersions and the resulting time-dependent drug release profile by dispersing, via hot-melt extrusion, fine drug particles in a thermodynamically stable crystalline state, or in a stabilized amorphous state into a polymeric carrier which will act to separate and isolate individual drug particles, thus preventing aggregation and agglomeration during processing and on storage.

### SUMMARY OF THE INVENTION

According to one aspect of the present invention, there is provided a hot melt extruded pharmaceutical composition comprising one or more active pharmaceutical ingredients and at least one water soluble or insoluble polymer or combination thereof, and one or more optional pharmaceutically acceptable excipients.

Where appropriate, each ingredient may be provided as the free base of or as its pharmaceutically acceptable salt, pharmaceutically acceptable solvate, pharmaceutically acceptable enantiomer, pharmaceutically acceptable derivative, pharmaceutically acceptable polymorph or pharmaceutically acceptable prodrug.

The hot melt extruded pharmaceutical composition is preferably provided as a solid oral pharmaceutical composition.

According to a second aspect of the present invention, there is provided a process of manufacturing the pharmaceutical composition by hot-melt extruding one or more active pharmaceutical ingredients and at least one water soluble or insoluble polymer or combination thereof, and one or more optional pharmaceutically acceptable excipients.

According to a third aspect of the invention, there is provided a melt extrusion process for manufacturing the solid oral pharmaceutical composition by melting one or more pharmaceutically active ingredients with or without at least one water soluble or insoluble polymer, wherein one component will melt and the other component will disperse in the melt thus forming a solid/glassy solution and/or suspension.

The mixing of the components can take place before, during, or after the formation of melt.

The active pharmaceutical ingredient is preferably selected from one or more of paracetamol, olanzapine, valsartan, clopidogrel, atorvastatin, simvastatin, amlodipine, ezetimibe, fenofibrate, voriconazole, topotecan, artesunate, amodiaquine, guggulosterone, ramipril, telmisartan, tibolone, tacrolimus, valacyclovir, valgancyclovir, estradiol, trenbolone, efavirenz, metformin, pseudoephedrine, verapamil, felodipine, valproic acid/sodium valproate, mesalamine, hydrochlorothiazide, levosulpiride, nelfinavir, cefixime and cefpodoxime proxetil.

It will be appreciated that each one of the active materials mentioned in the preceding paragraph may be provided, where appropriate, as the free base, or in the form of an appropriate pharmaceutically acceptable salt, a pharmaceutically acceptable solvate, a pharmaceutically acceptable enantiomer, a pharmaceutically acceptable derivative, a pharmaceutically acceptable polymorph, pharmaceutically acceptable ester or a pharmaceutically acceptable prodrug thereof. Thus, throughout this specification, references to an active material should, therefore, be read as including, where appropriate, the free base, or in the form of an appropriate pharmaceutically acceptable salt, a pharmaceutically acceptable solvate, a pharmaceutically acceptable enantiomer, a pharmaceutically acceptable derivative, a pharmaceutically acceptable polymorph, pharmaceutically acceptable ester or a pharmaceutically acceptable prodrug thereof.

### DETAILED DESCRIPTION OF THE INVENTION

As discussed above and hereinafter, the inventors have surprisingly found that the present invention can be formulated to achieve an advantageous dosage form comprising fine drug particles by dispersing fine drug particles in a thermodynamically stable crystalline state, or in a stabilized amorphous state into a polymeric carrier which will separate and isolate individual drug particles via hot-melt extrusion, thus preventing aggregation and agglomeration during processing and on storage.

The dosage form according to the present invention is characterized by excellent stability and in particular, exhibit high resistance against recrystallization or decomposition of the active ingredient(s).

Suitably, the formulations according to the invention are presented in solid dosage form, conveniently in unit dosage form, and include dosage form suitable for oral and/or buccal administration.

Solid dosage forms according to the present invention are preferably in the form of tablets, but other conventional dosages such as powders, pellets, capsules, and sachets may be provided.

A preferred formulation according to the invention is in tablet dosage form wherein one or more pharmaceutical active ingredients is combined with one or more water soluble or water insoluble polymer(s), or a combination thereof, and further one or more optional pharmaceutically acceptable excipients.

According to the present invention, the pharmaceutically active ingredients may be selected from, but not limited to, analgesics, anti-inflammatory, decongestants, hormones, anticancer drugs, antimalarials, antifungals, antipsychotics, antivirals, ACE inhibitors, Angiotensin II receptor blockers, HMG-Co reductase inhibitors, anti-hyperlipidemic agents, immunosuppressive drugs, antiplatelet agents, steroids, reverse transcriptase inhibitors, protease inhibitors, or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs and/or combination thereof.

Preferably, the pharmaceutically active ingredients of the present invention may be selected from, but not limited to, paracetamol, olanzapine, valsartan, clopidogrel, atorvastatin, simvastatin, amlodipine, ezetimibe, fenofibrate, voriconazole, topotecan, artesunate, amodiaquine, guggulosterone, ramipril, telmisartan, tibolone, tacrolimus, valacyclovir, valgancyclovir, estradiol, trenbolone, efavirenz, metformin, pseudoephedrine, verapamil, felodipine, valproic acid/sodium valproate, mesalamine, hydrochlorothiazide, levosulpiride, nelfinavir, cefixime, and cefpodoxime proxetil.

A tablet formulation is the preferred solid oral dosage form due to its greater stability, less risk of chemical interaction between different medicaments, smaller bulk, accurate dosage, and ease of production.

According to a preferred embodiment, the invention may be processed through hot-melt extrusion technique which involves hot-melt extrusion of one or more pharmaceutical active ingredient with one or more water soluble or water insoluble polymer(s) or a combination thereof.

The melt extrusion process is especially preferred for use with paracetamol, olanzapine, valsartan, clopidogrel, atorvastatin, simvastatin, amlodipine, ezetimibe, fenofibrate, voriconazole, topotecan, artesunate, amodiaquine, guggulosterone, ramipril, telmisartan, tibolone, tacrolimus, valacyclovir, valgancyclovir, estradiol, trenbolone, and efavirenz.

In general terms, the process of hot-melt extrusion is carried out in the conventional extruders as known to a person skilled in the art.

The melt-extrusion process comprises the steps of preparing a homogeneous melt of one or more drugs, the polymer and the excipients, and cooling the melt until it solidifies. “Melting” means a transition into a liquid or rubbery state in which it is possible for one component to get embedded homogeneously in the other.

Typically, one component will melt and the other components will dissolve in the melt, thus forming a solution. Melting usually involves heating above the softening point of the polymer. The preparation of the melt can take place in a variety of ways. The mixing of the components can take place before, during, or after the formation of the melt. For example, the components can be mixed first and then melt extruded, or be simultaneously mixed and melt extruded. Usually, the melt is homogenized in order to disperse the active ingredients efficiently. Also, it may be convenient first to melt the polymer and then to mix in and homogenize the active ingredients.

Usually, the melt temperature is in the range of about 70° C. to about 200° C., preferably from about 80° C. to about 180° C., most preferred from about 90° C. to about 150° C.

Suitable extruders include single screw extruders, intermeshing screw extruders or else multiscrew extruders, preferably twin screw extruders, which can be co-rotating or counter-rotating and, optionally, be equipped with kneading disks. It will be appreciated that the working temperatures will also be determined by the kind of extruder or the kind of configuration within the extruder that is used.

The extrudates can be in the form of beads, granulates, tube, strand, or cylinder and this can be further processed into any desired shape.

The term “extrudates” as used herein refers to solid product solutions, solid dispersions, and glass solutions of one or more drugs with one or more polymers and optionally pharmaceutically acceptable excipients.

According to a preferred embodiment, a powder blend of the one or more active drug(s) and polymers and optionally pharmaceutical excipients are transferred by a rotating screw of a single screw extruder through the heated barrel of an extruder whereby the powder blend melts and molten solution product is collected on a conveyor where it is allowed to cool to form an extrudate. Shaping of the extrudate can conveniently be carried out by a calendar with two counter—rotating rollers with mutually matching depressions on their surface.

A broad range of tablet forms can be attained by using rollers with different forms of depressions. Alternatively, the extrudate is cut into pieces after solidification and can be further processed into suitable dosage forms. More preferably, the extrudates thus finally obtained from the above process are then milled and ground to granules by the means known to a person skilled in the art.

Further, hot-melt extrusion is a fast, continuous, single pot manufacturing process without requirement of further drying or discontinuous process steps; it provides short thermal exposure of active allows processing of heat sensitive actives; process temperatures can be reduced by addition of plasticizers; comparatively lower investment for equipment as against other processes. The entire process is anhydrous and the intense mixing and agitation of the powder blend that occur during processing contribute to a very homogenous extrudate(s).

In one aspect, the preferred embodiment in accordance with the present invention may comprise one or more pharmaceutical active ingredient/s, one or more water soluble or water insoluble polymers which are melt extruded by the process as described herein, where a powder blend of one or more pharmaceutical active ingredient/s or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs and other excipients which may comprise suitable bulking agents and flavourants. These are so processed to form a powder blend which is transferred through the heated barrel of the extruder, whereby the powder blend melts and molten solution product is collected on a conveyor whereby it is allowed to cool and form an extrudate.

Alternatively, the extrudate is cut into pieces after solidification and can be further processed into suitable dosage forms. More preferably, the extrudates thus finally obtained from the above process are then milled and ground to granules by the means known to a person skilled in the art.

In one particularly preferred embodiment of the invention, valsartan with one or more water insoluble polymers are melt extruded by the process as described herein, to produce a powder blend of valsartan and one or more water soluble and other optional excipients, which may comprise suitable bulking agents, plasticizer and flavourants. As discussed above, the valsartan may be provided, where appropriate, as the free base, or in the form of an appropriate pharmaceutically acceptable salt, a pharmaceutically acceptable solvate, a pharmaceutically acceptable enantiomer, a pharmaceutically acceptable derivative, a pharmaceutically acceptable polymorph, pharmaceutically acceptable ester or a pharmaceutically acceptable prodrug thereof.

In another particularly preferred embodiment of the invention, clopidrogel with one or more water insoluble polymers and/or one or more water soluble polymers are melt extruded by the process as described herein, to produce a powder blend of clopidrogel and one or more water soluble and/or insoluble polymers and other optional excipients, which may comprise suitable bulking agents, plasticizer and flavourants. As discussed above, the clopidogrel may be provided, where appropriate, as the free base, or in the form of an appropriate pharmaceutically acceptable salt, a pharmaceutically acceptable solvate, a pharmaceutically acceptable enantiomer, a pharmaceutically acceptable derivative, a pharmaceutically acceptable polymorph, pharmaceutically acceptable ester or a pharmaceutically acceptable prodrug thereof.

In another particularly preferred embodiment of the invention, efavirenz with one or more water soluble polymers are melt extruded by the process as described herein, to produce a powder blend of efavirenz and one or more water soluble polymers and other optional excipients, which may comprise suitable bulking agents, plasticizer and flavourants. As discussed above, the efavirenz may be provided, where appropriate, as the free base, or in the form of an appropriate pharmaceutically acceptable salt, a pharmaceutically acceptable solvate, a pharmaceutically acceptable enantiomer, a pharmaceutically acceptable derivative, a pharmaceutically acceptable polymorph, pharmaceutically acceptable ester or a pharmaceutically acceptable prodrug thereof.

In another particularly preferred embodiment of the invention, olanzapine with one or more water soluble polymers are melt extruded by the process as described herein, to produce a powder blend of olanzapine and one or more water soluble polymer and other optional excipients, which may comprise suitable bulking agents, plasticizer and flavourants. As discussed above, the olanzapine may be provided, where appropriate, as the free base, or in the form of an appropriate pharmaceutically acceptable salt, a pharmaceutically acceptable solvate, a pharmaceutically acceptable enantiomer, a pharmaceutically acceptable derivative, a pharmaceutically acceptable polymorph, pharmaceutically acceptable ester or a pharmaceutically acceptable prodrug thereof.

In another particularly preferred embodiment of the invention, voriconazole with one or more water soluble polymers are melt extruded by the process as described herein, to produce a powder blend of voriconazole and one or more water soluble polymers and other optional excipients, which may comprise suitable bulking agents, plasticizer and flavourants. As discussed above, the voriconazole may be provided, where appropriate, as the free base, or in the form of an appropriate pharmaceutically acceptable salt, a pharmaceutically acceptable solvate, a pharmaceutically acceptable enantiomer, a pharmaceutically acceptable derivative, a pharmaceutically acceptable polymorph, pharmaceutically acceptable ester or a pharmaceutically acceptable prodrug thereof.

In another particularly preferred embodiment of the invention, valgancyclovir with one or more water soluble polymers and/or one or more water insoluble polymers are melt extruded by the process as described herein, to produce a powder blend of valgancyclovir and one or more water soluble polymers and/or one or more water insoluble polymers and other optional excipients, which may comprise suitable bulking agents, plasticizer and flavourants. As discussed above, the valgancyclovir may be provided, where appropriate, as the free base, or in the form of an appropriate pharmaceutically acceptable salt, a pharmaceutically acceptable solvate, a pharmaceutically acceptable enantiomer, a pharmaceutically acceptable derivative, a pharmaceutically acceptable polymorph, pharmaceutically acceptable ester or a pharmaceutically acceptable prodrug thereof.

These are so processed to form a powder blend which is transferred through the heated barrel of the extruder, whereby the powder blend melts and molten solution product is collected on a conveyor whereby it is allowed to cool and form an extrudate.

Alternatively, the extrudate is cut into pieces after solidification and can be further processed into suitable dosage forms. More preferably, the extrudates thus finally obtained from the above process are then milled and ground to granules by the means known to a person skilled in the art.

In a still alternative process, the present invention may further be allowed to form granules which may be compressed to form tablets, or the granules may be filled into capsules, sachets, or in a similar dosage form.

This process involves heating the polymer(s) to soften it, without melting it, and mixing the active ingredient(s) with polymer(s), to form granules of the or each active ingredient dispersed in the or each polymer. In this alternative process, unlike the hot-melt extrusion process, the polymer(s) is not melted to form a liquid in which the active ingredient(s) are dissolved or dispersed. Instead, the polymer remains solid, but is sufficiently soft to allow the active material(s) to be mixed therewith and distributed throughout the polymer(s). The process can be carried out in the same type of extrusion apparatus as the hot melt extrusion process, except that the product is not extruded through the extrusion nozzle of the apparatus.

This will yield uniform and compact granules. It will be readily acknowledged by the person skilled in the art that the said process may be applicable to pharmaceutical active ingredients as mentioned throughout the specification. However, this process is particularly suitable for the preparation of pharmaceutical compositions comprising one or more of metformin, pseudoephedrine, verapamil, felodipine, valproic acid/sodium valproate, mesalamine, hydrochlorthiazide, levo sulpiride, efavirenz, nelfinavir and antibiotics like cephalosporins, e.g., cefixime, cefpodoxime proxetil.

Verapamil with one or more water soluble polymers and/or one or more water insoluble polymers may be produced by this hot granulation process. The polymer(s) is softened, but not heated to produce a liquid form, and mixed with the verapimil and other optional excipients, which may comprise suitable bulking agents, plasticizer and flavourants, and processed to produce granules of the verapimil and any excipients dispersed in the polymer(s). As discussed above, the verapamil may be provided, where appropriate, as the free base, or in the form of an appropriate pharmaceutically acceptable salt, a pharmaceutically acceptable solvate, a pharmaceutically acceptable enantiomer, a pharmaceutically acceptable derivative, a pharmaceutically acceptable polymorph, pharmaceutically acceptable ester or a pharmaceutically acceptable prodrug thereof.

The water soluble polymers that can be used, according to the present invention, comprises of homopolymers and co-polymers of N-vinyl lactams, especially homopolymers and co-polymers of N-vinyl pyrrolidone, e.g., polyvinylpyrrolidone (PVP), co-polymers of PVP and vinyl acetate, co-polymers of N-vinyl pyrrolidone and vinyl acetate or vinyl propionate, dextrins such as grades of maltodextrin, cellulose esters and cellulose ethers, high molecular polyalkylene oxides, such as polyethylene oxide and polypropylene oxide, and co-polymers of ethylene oxide and propylene oxide. The water soluble polymer is preferably present in the range wherein the ratio of drug to polymer is 1:0.5 to 1:6.

The water insoluble polymers that can be used, according to the present invention, comprises of acrylic copolymers, e.g., Eudragit E100 or Eudragit EPO; Eudragit L30D-55, Eudragit FS30D, Eudragit RL30D, Eudragit RS30D, Eudragit NE30D, Acryl-Eze (Colorcon Co.); polyvinylacetate, for example, Kollicoat SR 30D (BASF Co.); cellulose derivatives such as ethylcellulose, cellulose acetate, e.g., Surelease (Colorcon Co.), Aquacoat ECD and Aquacoat CPD (FMC Co.). The most preferred water insoluble polymer is Eudragit E100. The water insoluble polymer is preferably present in the range wherein the ratio of drug to polymer is 1:1 to 1:6. Additionally, the water insoluble polymer may be combined with organic acids, such as from citric acid, tartaric acid, glycolic acid, etc.

Plasticizers can be incorporated depending on the polymer and the process requirement. These, advantageously, when used in the hot-melt extrusion process decrease the glass transition temperature of the polymer. Plasticizers also help in reducing the viscosity of the polymer melt, and thereby allow for lower processing temperature and extruder torque during hot-melt extrusion. Examples of plasticizers which can be used in the present invention, include, but are not limited to, polysorbates such as sorbitan monolaurate (Span 20), sorbitan monopalmitate, sorbitan monostearate, sorbitan monoisostearate; citrate ester type plasticizers like triethyl citrate, citrate phthalate; propylene glycol; glycerin; polyethylene glycol (low and high molecular weight); triacetin; dibutyl sebacate, tributyl sebacate; dibutyltartrate, dibutyl phthalate. The plasticizer is preferably present in an amount ranging from 0% to 10% to the weight of polymer.

The present invention may comprise suitable disintegrating agents which includes, but are not limited to, croscarmellose sodium, crospovidone, sodium starch glycolate, corn starch, potato starch, maize starch and modified starches, calcium silicates, low substituted hydroxy-propylcellulose. The amount of disintegrating agent is preferably in the range of 5% to 35% by weight of the composition.

The present invention may further comprise suitable bulking agents which includes, but are not limited to, saccharides, including monosaccharides, disaccharides, polysaccharides and sugar alcohols such as arabinose, lactose, dextrose, sucrose, fructose, maltose, mannitol, erythritol, sorbitol, xylitol, lactitol, and other bulking agents such as powdered cellulose, microcrystalline cellulose, purified sugar and derivatives thereof. The formulation may incorporate one or more of the above bulking agents. The amount of the bulking agent is preferably in the range of 15% to 70% by weight of the composition.

The present invention may further incorporate suitable lubricants and glidants which may include, but are not limited to, stearic acid and its derivatives or esters like sodium stearate, magnesium stearate and calcium stearate and the corresponding esters such as sodium stearyl fumarate; talc and colloidal silicon dioxide respectively. The amount of lubricant and/or glidant is preferably in the range of 0.25% to 5% by weight of the composition.

According to the present invention, the tablet may be seal coated. Preferably, the tablet may be seal coated and finally film coated. The formulation can be coated with Ready colour mix systems (such as Opadry colour mix systems).

According to one embodiment, valsartan and one or more excipients which includes, but are not limited to, polymers (i.e., combination of water soluble and water insoluble), one or more plasticizer, one or more disintegrants, one or more lubricants and glidants are extruded through hot-melt extrusion technique wherein extrudates are obtained which can be molded into desired shapes that can be filled in sachets/capsules or can be granulated. Alternatively, the granules may be compressed into tablets.

According to a second embodiment, olanzapine and one or more excipients which includes, but are not limited to, polymers (i.e., water soluble), one or more plasticizer, one or more disintegrants, one or more lubricants and glidants are extruded through hot-melt extrusion technique wherein extrudates are obtained which can be molded into desired shapes that can be filled in sachets/capsules or can be granulated. Alternatively, the granules may be compressed into tablets.

According to a third embodiment, voriconazole and one or more excipients which includes, but are not limited to, polymers (i.e., water soluble), one or more plasticizer, one or more disintegrants, one or more lubricants and glidants are extruded through hot-melt extrusion technique wherein extrudates are obtained which can be molded into desired shapes that can be filled in sachets/capsules or can be granulated. Alternatively, the granules may be compressed into tablets.

According to a fourth embodiment, valgancyclovir and one or more excipients which includes, but are not limited to, polymers (i.e., water soluble and/or water insoluble), one or more plasticizer, one or more disintegrants, one or more lubricants and glidants are extruded through hot-melt extrusion technique wherein extrudates are obtained which can be molded into desired shapes that can be filled in sachets/capsules or can be granulated. Alternatively, the granules may be compressed into tablets.

According to another aspect of the invention, a process for making a pharmaceutical composition comprising heating one or more polymer to soften it, without melting it, and mixing one or more active ingredient with the polymer(s), to form granules of the active ingredient(s) dispersed in the or each polymer(s).

The exact temperature is not critical. What is important is that the active material does not degrade at the temperature used, and that the extruder is capable of processing the polymer material, in a soft, but solid, form, together with the active material to provide a dispersion of the active material in the polymer material. The preferred temperature for this process is from 30° C. to 120° C.

In accordance with this aspect of the invention, it is possible to obtain uniform and compact granules. The granules as obtained in this way may be further mixed, sieved, sifted and compressed into a single tablet or may be filled into capsules or sachets or the granules may be administered directly. The tablet may be seal coated and/or film coated.

Alternatively, in a suitable pharmaceutical dosage form comprising two actives, according to the present invention, the or each granules (comprising the individual actives) as obtained above may be individually compressed into two tablets and finally compacted and compressed into a bilayer tablet. The tablet may be seal coated and finally film coated.

The formulation can be coated with Ready colour mix systems (such as Opadry colour mix systems).

The administration of the formulation/composition according to the present invention may be envisaged to cover different unit dosage formulations including suspensions, capsules, tablets, sachets, solutions, dry syrups, emulsions containing conventional non-toxic pharmaceutically acceptable carriers, adjuvants and vehicles.

### EXAMPLES

The following examples are for the purpose of illustration of the invention only and are not intended in any way to limit the scope of the present invention.

### Example 1

| Sr. No. | INGREDIENTS | Qnty/tab (mg) |
|---|---|---|
| 1. | Valgancyclovir hydrochloride | 496.30 |
| 2. | Kollidon VA-64 | 450.00 |
| 3. | Sorbitan monolaurate (Span 20) | 22.50 |

Extragranular

| | | |
|---|---|---|
| 4. | Microcrystalline cellulose | 105.20 |
| 5. | Crospovidone | 20.00 |
| 6. | Magnesium stearate | 6.00 |

Film Coating

| | | |
|---|---|---|
| 7. | Ready colour mix system | 15.00 |
| 8. | Purified water | q. s. |
| | Total | 1115.00 |

(1) Valgancyclovir was sifted and mixed together small amount of Kollidon VA 64 and Span 20 in a mixer.

(2) The contents obtained in (1) were mixed and finally subjected to hot-melt extrusion (HME) wherein the melting temperature for the extrusion process ranges from 70 to 200° C., with the molten mass thus obtained was collected on a conveyor where it was cooled to form extrudates and these extrudates on further milling were converted into granules which was followed by addition of microcrystalline cellulose and crospovidone and further lubricated with magnesium stearate.

(3) The granules obtained in (2) were compressed to form a tablet which was finally coated with ready colour mix system.

### Example 2

| Sr. No. | INGREDIENTS | Qnty/tab (mg) |
|---|---|---|
| 1. | Valgancyclovir hydrochloride | 496.30 |
| 2. | Eudragit E 100 | 450.00 |
| 3. | Eudragit NE 30D | 22.50 |

Extragranular

| | | |
|---|---|---|
| 4. | Microcrystalline cellulose | 105.20 |
| 5. | Crospovidone | 20.00 |
| 6. | Sodium stearyl fumarate | 6.00 |

Film Coating

| | | |
|---|---|---|
| 7. | Ready colour mix system | 15.00 |
| 8. | Purified water | q. s. |
| | Total | 1115.00 |

(1) Valgancyclovir was sifted and mixed together small amount of Eudragit E100 & Eudragit NE 30D in a mixer.

(2) The contents obtained in (1) were mixed and finally subjected to hot-melt extrusion (HME) wherein the melting temperature for the extrusion process ranges from 70 to 200° C., with the molten mass thus obtained was collected on a conveyor where it was cooled to form extrudates and these extrudates on further milling were converted into granules which was followed by addition of microcrystalline cellulose and crospovidone and further lubricated with sodium stearyl fumarate.

(3) The granules obtained in (2) were compressed to form a tablet which was finally coated with ready colour mix system.

### Example 3

| Sr. No. | Ingredients | Qty/Tab (mg) |
|---|---|---|
| 1 | Efavirenz | 200.00 |
| 2 | Kollidon VA-64 | 200.00 |
| 3 | Colloidal silicon dioxide | 10.00 |

Extragranular

| | | |
|---|---|---|
| 4 | Lactose | 65.00 |
| 5 | Sodium starch glycolate | 20.00 |
| 6 | Magnesium stearate | 5.00 |

Film coating

| | | |
|---|---|---|
| 7 | Ready colour mix system | 15.00 |
| 8 | Purified water | q. s. |
| | Total | 515.00 |

(1) Efavirenz was sifted and mixed together small amount of Kollidon VA 64 and colloidal silicon dioxide in a mixer.

(2) The contents obtained in (1) were mixed and finally subjected to hot-melt extrusion (HME) wherein the melting temperature for the extrusion process ranges from 70 to 200° C., with the molten mass thus obtained was collected on a conveyor where it was cooled to form extrudates and these extrudates on further milling were converted into granules which was followed by addition of lactose and sodium starch glycolate and further lubricated with magnesium stearate.

(3) The granules obtained in (2) were compressed to form a tablet which was finally coated with ready colour mix system.

### Example 4

| Sr. No. | Ingredients | Qty/Tab (mg) |
|---|---|---|
| 1 | Efavirenz | 600.00 |
| 2 | Kollidon VA-64 | 600.00 |
| 3 | Colloidal silicon dioxide | 10.00 |

Extragranular

| | | |
|---|---|---|
| 4 | Lactose | 150.00 |
| 5 | Microcrystalline cellulose | 150.00 |
| 6 | Sodium carboxy methyl cellulose | 30.00 |
| 7 | Magnesium stearate | 10.00 |

Film coating

| | | |
|---|---|---|
| 8 | Ready colour mix system | 15.00 |
| 9 | Purified water | q. s. |
| | Total | 1565.00 |

(1) Efavirenz was sifted and mixed together small amount of Kollidon VA 64 and colloidal silicon dioxide in a mixer.

(2) The contents obtained in (1) were mixed and finally subjected to hot-melt extrusion (HME) wherein the melting temperature for the extrusion process ranges from 70 to 200° C., with the molten mass thus obtained was collected on a conveyor where it was cooled to form extrudates and these extrudates on further milling were converted into granules which was followed by addition of lactose, microcrystalline cellulose, sodium carboxy methyl cellulose and further lubricated with magnesium stearate.

(3) The granules obtained in (2) were then coated with ready colour mix system and finally filled in capsules.

### Example 5

| Sr. No. | Ingredients | Qty/Tab (mg) |
|---|---|---|
| 1 | Clopidogrel bisulphate | 97.854 |
| 2 | Kollidon VA 64 | 195.00 |
| 3 | Colloidal silicon dioxide | 5.00 |
| 4 | Atorvastatin calcium | 80.00 |
| 5 | Kollidon VA64 | 400.00 |
| 6 | Colloidal silicon dioxide | 5.00 |
| 7 | Span 20 | 17.146 |
| 8 | Mannitol SD 200 | 280.00 |
| 9 | Calcium carbonate | 20.00 |
| 10 | Hydroxy propyl cellulose (LHPC) | 80.00 |
| 11 | Calcium stearate | 15.00 |
| 12 | Talc | 5.00 |

Coating

| | | |
|---|---|---|
| 13 | Ready colour mix system | 15.00 |
| 14 | Purified water | q. s. |
| | Total | 1015.00 |

(1) Clopidrogel bisulphate was mixed with pre-sieved and pre-sifted amounts of Kollidon VA64 and colloidal silicon dioxide.

(2) Atorvastatin calcium with small amount of colloidal silicon dioxide was sifted and mixed together with Kollidon VA 64 and Span 20 in a mixer.

(3) The contents obtained in (1) and (2) were finally subjected to hot-melt extrusion (HME) separately wherein the melting temperature for the extrusion process ranges from 70 to 200° C., with the molten mass thus obtained was collected on a conveyor where it was cooled to form extrudates and these extrudates on further milling were converted into granules which was followed by addition of mannitol, Hydroxy propyl cellulose (LHPC), calcium carbonate and talc and further lubricated with calcium stearate.

(4) The granules obtained in (3) were compressed together to form a tablet which was finally coated with ready colour mix system.

### Example 6

| Sr. No. | Ingredients | Qty/Tab (mg) |
|---|---|---|
| 1 | Olanzapine | 10.00 |
| 2 | Kollidon VA-64 | 20.00 |
| 3 | Sorbitan monolaurate (Span 20) | 0.50 |

Extragranular

| | | |
|---|---|---|
| 4 | Microcrystalline cellulose | 320.50 |
| 5 | Hydroxypropyl cellulose | 45.00 |
| 6 | Magnesium stearate | 4.00 |

Film coating

| | | |
|---|---|---|
| 7 | Ready colour mix system | 15.00 |
| 8 | Purified water | q. s. |
| | Total | 415.00 |

(1) Olanzapine was sifted and mixed together small amount of Kollidon VA 64 and Span 20 in a mixer.

(2) The contents obtained in (1) were mixed and finally subjected to hot-melt extrusion (HME) wherein the melting temperature for the extrusion process ranges from 70 to 200° C., with the molten mass thus obtained was collected on a conveyor where it was cooled to form extrudates and these extrudates on further milling were converted into granules which was followed by addition of microcrystalline cellulose and hydroxypropyl cellulose and further lubricated with magnesium stearate.

(3) The granules obtained in (2) were compressed to form a tablet which was finally coated with ready colour mix system.

### Example 7

| Sr. No. | Ingredients | Qty/Tab (mg) |
|---|---|---|
| 1 | Olanzapine | 10.00 |
| 2 | Eudragit E100 | 40.00 |
| 3 | Eudragit NE 30D | 2.00 |

Extragranular

| | | |
|---|---|---|
| 4 | Microcrystalline cellulose | 299.00 |
| 5 | Hydroxypropyl cellulose | 45.00 |
| 6 | Magnesium stearate | 4.00 |

Film coating

| | | |
|---|---|---|
| 7 | Ready colour mix system | 15.00 |

(1) Olanzapine was sifted and mixed together small amount of Eudragit E100 and Eudragit NE 30D in a mixer.

(2) The contents obtained in (1) were mixed and finally subjected to hot-melt extrusion (HME) wherein the melting temperature for the extrusion process ranges from 70 to 200° C., with the molten mass thus obtained was collected on a conveyor where it was cooled to form extrudates and these extrudates on further milling were converted into granules which was followed by addition of microcrystalline cellulose and hydroxypropyl cellulose and further lubricated with magnesium stearate.

(3) The granules obtained in (2) were compressed to form a tablet which was finally coated with ready colour mix system.

### Example 8

| Sr. No. | Ingredients | Qty/Tab (mg) |
|---|---|---|
| 1 | Olanzapine | 10.00 |
| 2 | Maltodextrin | 45.00 |
| 3 | PEG 6000 | 5.00 |

Extragranular

| | | |
|---|---|---|
| 4 | Microcrystalline cellulose | 291.00 |
| 5 | Hydroxypropyl cellulose | 45.00 |
| 6 | Magnesium stearate | 4.00 |

Film Coating

| | | |
|---|---|---|
| 7 | Ready Colour Mix system | 15.00 |
| 8 | Purified Water | q. s. |
| | Total | 415.00 |

(1) Olanzapine was sifted and mixed together small amount of maltodextrin and PEG 6000 in a mixer.

(2) The contents obtained in (1) were mixed and finally subjected to hot-melt extrusion (HME) wherein the melting temperature for the extrusion process ranges from 70 to 200° C., with the molten mass thus obtained was collected on a conveyor where it was cooled to form extrudates and these extrudates on further milling were converted into granules which was followed by addition of microcrystalline cellulose and hydroxypropyl cellulose and further lubricated with magnesium stearate.

(3) The granules obtained in (2) were compressed together to form a tablet which was finally coated with ready colour mix system.

### Example 9

| Sr. No. | Ingredients | Qty/Tab (mg) |
|---|---|---|
| 1 | Voriconazole | 50.00 |
| 2 | Kollidon VA 64 | 150.00 |

Extragranular

| | | |
|---|---|---|
| 3 | Croscarmellose sodium | 50.00 |
| 4 | Microcrystalline cellulose | 199.25 |
| 5 | Magnesium stearate | 0.75 |

Film Coating

| | | |
|---|---|---|
| 6 | Ready Colour Mix system | 15.00 |
| 7 | Purified Water | q. s. |
| | Total | 465.00 |

(1) Voriconazole was sifted and mixed together small amount of Kollidon VA 64 in a mixer.

(2) The contents obtained in (1) were mixed and finally subjected to hot-melt extrusion (HME) wherein the melting temperature for the extrusion process ranges from 70 to 200° C., with the molten mass thus obtained was collected on a conveyor where it was cooled to form extrudates and these extrudates on further milling were converted into granules which was followed by addition of microcrystalline cellulose and croscarmellose sodium and further lubricated with magnesium stearate.

(3) The granules obtained in (2) were compressed together to form a tablet which was finally coated with ready colour mix system.

### Example 10

| | | |
|---|---|---|
| Sr No. | Ingredients | Qty (mg/tab) |

Dry mix

| | | |
|---|---|---|
| 1. | Verapamil hydrochloride | 240.00 |
| 2. | Sodium alginate | 180.00 |
| 3. | Microcrystalline cellulose | 169.00 |
| 4. | Polyvinyl pyrrolidone K30 (PVP K30) | 33.00 |
| 5. | Colloidal silicon dioxide | 5.00 |

Lubrication

| | | |
|---|---|---|
| 6. | Magnesium stearate | 3.00 |
| | Total | 360.00 |

Film Coating

| | | |
|---|---|---|
| 7. | Opadry 04F86549 Brown | 10.00 |
| 8. | Purified water | q. s. |
| | Total | 370.00 |

(1) Verapamil hydrochloride was sifted and mixed with sodium alginate and microcrystalline cellulose to form uniform blend with Povidone K30 and colloidal silicon.

(2) The blend obtained above was passed through twin screw extruder maintained at a temperature about 30° C. to 120° C. and the granules formed were lubricated with magnesium stearate.

(3) The granules obtained were compressed to form tablets which were finally film coated.

### Example 11

| | | |
|---|---|---|
| Sr. No. | Ingredients | Qty (mg/tab) |

Intragranulation

| | | |
|---|---|---|
| 1. | Metformin Hydrochloride | 500.0 |
| 2. | Microcrystalline cellulose | 100.0 |
| 3. | Hypromellose (HPMC K 100 M) | 150.0 |
| 4. | Carboxy Methyl cellulose sodium | 125.0 |
| 5. | Colloidal silicon Dioxide | 3.0 |

Intragranular lubrication

| | | |
|---|---|---|
| 6. | Magnesium Stearate | 0.80 |

Extragranulation

| | | |
|---|---|---|
| 7. | Colloidal silicon Dioxide | 4.0 |
| 8. | Microcrystalline cellulose | 163.2 |

Lubrication

| | | |
|---|---|---|
| 9. | Magnesium Stearate | 4.0 |
| | Total | 1050.0 |

(1) Metformin Hydrochloride was sifted and mixed with pre-sifted quantities of microcrystalline cellulose, hypromellose, carboxymethyl cellulose sodium and colloidal silicon dioxide to form a uniform blend.

(2) The above blend was lubricated with magnesium stearate and granulated in twin screw extruder.

(3) Rest quantity of colloidal silicon dioxide and microcrystalline cellulose was added followed by magnesium stearate.

(4) The granules obtained above were finally compressed into tablets.

### Example 12

| | | |
|---|---|---|
| Sr. No. | Ingredients | Qty (mg/tab) |

Intragranulation

| | | |
|---|---|---|
| 1. | Pseudoephedrine hydrochloride | 120.00 |
| 2. | Lactose monohydrate | 34.50 |
| 3. | Hypromellose (HPMC K4M) | 85.00 |
| 4. | Hypromellose (HPMC K15M) | 95.00 |
| 5. | Colloidal silicon dioxide | 1.50 |

Intragranular lubrication

| | | |
|---|---|---|
| 6. | Magnesium Stearate | 0.50 |

Lubrication

| | | |
|---|---|---|
| 7. | Talc | 2.00 |
| 8. | Colloidal silicon dioxide | 1.50 |
| 9. | Magnesium stearate | 3.00 |
| | Total | 343.0 |

(1) Pseudoephedrine hydrochloride was sifted and mixed with pre-sifted quantities of lactose monohydrate, microcrystalline cellulose, hypromellose, colloidal silicon dioxide to form uniform blend followed by lubrication with magnesium stearate.

(2) The above blend was granulated in twin screw extruder to form granules followed by addition of talc and rest quantities of colloidal silicon dioxide and magnesium stearate.

(3) The granules obtained above were finally compressed into tablets.

### Example 13

| | | |
|---|---|---|
| Sr. No. | Ingredients | Qty (mg/tab) |

Premix

| | | |
|---|---|---|
| 1. | Felodipine | 2.50 |
| 2. | Lactose monohydrate | 25.20 |
| 3. | Microcrystalline cellulose | 51.60 |
| 4. | Propyl Gallate | 0.067 |
| 5. | Povidone K-30 | 7.35 |
| 6. | Hydroxypropylmethylcellulose E 50 | 55.00 |
| | (HPMC E 50) | |

Blending & Lubrication

| | | |
|---|---|---|
| 7. | Hydroxypropylmethylcellulose E 50 | 55.00 |
| | (HPMC E 50) | |
| 8. | Colloidal silicon dioxide | 1.45 |
| 9. | Microcrystalline cellulose | 8.00 |
| 10. | Magnesium stearate | 0.833 |
| | Total | 207.00 |

Coating

| | | |
|---|---|---|
| 11. | Hydroxypropylmethylcellulose 6 cps | 6.66 |
| | (HPMC 6 cps) | |
| 12. | Propylene glycol | 1.165 |
| 13. | Red oxide of iron | 0.01 |
| 14. | Ferric oxide yellow | 0.007 |
| 15. | Titanium dioxide | 0.435 |
| 16. | Talc | 1.008 |
| 17. | Purified water | q. s. |
| | Total | 216.00 |

(1) Felodipine was sifted and mixed with pre-sifted quantities of lactose monohydrate, microcrystalline cellulose, propyl gallate, Povidone K 30, and HPMC E 50 to form uniform blend.

(2) The above blend was granulated in twin screw extruder maintained at a temperature about 30° C. to 120° C. to form granules followed by mixing with HPMC E 50, colloidal silicon dioxide, microcrystalline cellulose and magnesium stearate.

(3) The granules obtained above were finally compressed into tablets and finally coated.

### Example 14

| Sr. No. | INGREDIENTS | Qnty/tab (mg) |
|---|---|---|
| 1. | Paracetamol | 80.00 |
| 2. | Eudragit E 100 | 40.00 |
| 3. | Stearic acid | 5.00 |
| 4. | Tartaric acid | 5.00 |

Blending & Lubrication

| | | |
|---|---|---|
| 5. | Sorbitol | 50.00 |
| 6. | Mannitol | 229.25 |
| 7. | Crospovidone | 22.50 |
| 8. | FD&C colourants | 0.50 |
| 9. | Strawberry flavour | 3.00 |
| 10. | Xylitol | 5.00 |
| 11. | Sucralose | 3.00 |
| 12. | Magnesium stearate | 6.75 |
| | Total | 450.00 |

(1) Paracetamol was sifted and mixed together with Eudragit E 100, stearic acid and tartaric acid in a mixer.

(2) The contents obtained in (1) were subjected to hot-melt extrusion (HME) wherein the melting temperature for the extrusion process ranges from 80 to 140° C., with the molten mass thus obtained was collected on a conveyor where it was cooled to form extrudates and these extrudates on further milling were converted into granules which was followed by addition of sorbitol, Mannitol, crospovidone, xylitol, sucralose, strawberry flavour, FD&C colourant and further lubricated with magnesium stearate.

(3) The granules obtained in (2) were compressed into tablets.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “a polymer” includes a single polymer as well as two or more different polymers; reference to a “plasticizer” refers to a single plasticizer or to combinations of two or more plasticizer, and the like.

1. A pharmaceutical composition comprising a solid unit dosage form comprising: one or more of pharmaceutically active ingredients selected from paracetamol, olanzapine, valsartan, clopidogrel, atorvastatin, simvastatin, amlodipine, ezetimibe, fenofibrate, voriconazole, topotecan, artesunate, amodiaquine, guggulosterone, ramipril, telmisartan, tibolone, tacrolimus, valacyclovir, valgancyclovir, estradiol, trenbolone, efavirenz, metformin, pseudoephedrine, verapamil, felodipine, valproic acid/sodium valproate, mesalamine, hydrochlorothiazide, levosulpiride, nelfinavir, cefixime and cefpodoxime proxetil in combination with a water insoluble polymer and/or a water soluble polymer.

2. The pharmaceutical composition according to claim 1, wherein the ratio of the weight of the pharmaceutically active ingredient(s) to the weight of the polymer(s) is from 1:0.5 to 1:6.

3. The pharmaceutical composition according to claim 1, wherein the pharmaceutically active ingredient(s) is respectively dispersed in, or dissolved in, the polymer(s).

4. The pharmaceutical composition according to claim 3, wherein at least one pharmaceutically acceptable excipients is dispersed in, or dissolved in, the polymers.

5. The pharmaceutical composition according to claim 2, which is obtainable by hot melt extruding said pharmaceutically active ingredient(s) with the polymer(s).

6. The pharmaceutical composition according to claim 2, which is obtainable by heating the polymer(s) to soften it, without melting it, and mixing the or active ingredient(s) with polymer(s) to form granules of the or each active ingredient(s) dispersed in the polymer(s).

7. The process for making a pharmaceutical composition as defined in claim 1, comprising hot melt extruding at least one of the pharmaceutically active ingredients with the polymer(s) to form an extrudate, then formulating the extrudate into a pharmaceutical composition.

8. The process according to claim 7, wherein the pharmaceutically active material(s) is mixed with the water soluble polymer and/or a water insoluble polymer prior the hot melt extrusion step.

9. The process according to claim 7, comprising preparing a substantially homogeneous melt of the pharmaceutically active ingredient(s), the polymer(s) and optionally one or more pharmaceutically acceptable excipients, extruding the melt, and cooling the melt until it solidifies.

10. The process according to claim 9, wherein the melt is formed at a temperature from substantially 50°-C to substantially 200°-C.

11. The process according to claim 7, wherein the or each active pharmaceutical ingredient, the polymer, and, optionally, one or more pharmaceutically acceptable excipients are processed to form a powder blend which is transferred through the heated barrel of the extruder, whereby the powder blend melts and a molten solution product is formed, which is allowed to cool to form an extrudate.

12. The process according to claim 11, comprising formulating the cooled extrudate into a desired pharmaceutical dosage form.

13. The process according to claim 7, wherein the pharmaceutically active ingredient is selected from one or more of paracetamol, olanzapine, valsartan, clopidogrel, atorvastatin, simvastatin, amlodipine, ezetimibe, fenofibrate, voriconazole, topotecan, artesunate, amodiaquine, guggulosterone, ramipril, telmisartan, tibolone, tacrolimus, valacyclovir, valgancyclovir, estradiol, trenbolone and efavirenz.

14. The process for making a pharmaceutical composition as defined in claim 1, comprising heating the polymer(s) to soften it, without melting it, and mixing the active ingredient(s) with polymer(s), to form granules of the or each active ingredient dispersed in the or each polymer.

15. The process according to claim 14, wherein the temperature is in the range 30° C. to 120° C.

16. The process according to claim 14, further comprising allowing the granules to cool, then formulating them into a desired pharmaceutical dosage form.

17. The process according to claim 13, wherein the pharmaceutically active ingredient is selected from one or more of efavirenz, metformin, pseudoephedrine, verapamil, felodipine, valproic acid/sodium valproate, mesalamine, hydrochlorothiazide, levosulpiride, nelfinavir, cefixime and cefpodoxime proxetil.

18. The pharmaceutical dosage form made according to the process of claim 7, in the form of a tablet or capsule.
